(19) United States
(12) Patent Application Publication
(10) Pub. No.: US 2007/0059714 A1
(43) Pub. Date: Mar. 15, 2007

(54) Detection of presence and antibiotic susceptibility of enterococci

(76) Inventors: Birgit Strommenger, Wernigerode (DE); Christiane Kettlitz, Wernigerode (DE); Ulrich Nubel, Wernigerode (DE); Guido Werner, Wernigerode (DE); Ingo Klare, Wernigerode (DE); Wolfgang Witte, Wernigerode (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/224,393

(22) Filed: Sep. 12, 2005

(51) Int. Cl. C12Q 1/68; C12M 1/34

(52) U.S. Cl. 435/6; 435/287.2

(57) ABSTRACT

The present invention relates in general to the detection of antibiotic resistance genes in Enterococci. The present invention discloses a micro-array for the detection of the presence of bacteria of the genus Enterococcus and antibiotic resistance genes in said organism, a method for the detection of said genes and a kit. This micro-array concept offers the rapid sensitive and specific identification of antibiotic resistance profiles.

### BACKGROUND OF THE INVENTION

1. Field of the Invention

The present invention relates in general to the detection of Enterococci (bacteria of the genus Enterococcus) exhibiting multi-resistance to antibiotics. In particular, the present invention pertains to a micro-array for the detection of target genes conferring antibiotic resistance to bacteria of said genus, a method for the detection of the target genes and a kit. The present micro-array offers a rapid, sensitive and specific identification of antibiotic resistance profiles of biological samples. Due to the emerging broadening of antibiotic resistance, it may be adapted to the respective changed clinical and epidemiological requirements in clinical diagnosis as well as in epidemiological studies.

2. Description of the Related Art

Enterococci are part of the normal flora of both, the human and the animal gastrointestinal tract. Bacteria of the genus Enterococcus are opportunistic pathogens associated with urinary tract infections, blood stream infections and endocarditis. In case of an endocarditis, the bacteria grow in the heart valves of an infected patient and cause damage thereto. Endocarditis is currently diagnosed by clinical symptoms, echocardiogram and the presence of heart murmurs. The causative microorganism is usually identified by blood culture (culture-positive endocarditis). However, in approximately 10% of infective endocarditis patients the blood culture is negative. This may lead to both a wrong diagnosis and delayed treatment.

Several possibilities for the treatment and/or prevention of infections with Enterococci are known in the art. Since infections with said bacteria may be mediated by the intestine, non-detrimental microorganisms capable to supersede and to colonize the intestine, such as probiotic lactic acid bacteria, may be used for the prevention or alleviation of Enterococcus infections. For example in U.S. Pat. No. 6,524,574 such a mixture of probiotics and yeasts effective to reduce the contamination of enteric bacteria in humans and other monogastric animals is disclosed.

Another regimen is specified in WO 94/15640, which is reported to be useful in the treatment of acute infections of e.g. open wounds. This regimen is based on compositions containing several immunoglobulins, which are applied directly on the source of infection.

Most common, however, is the administration of an antibiotic or a combination therapy with different antibiotics. Familiar antibiotics used are selected from compound classes like macrolide, lincosamide and streptogramin (MLS) antibiotics, which are chemically distinct inhibitors of bacterial protein synthesis.

Bacteriostatic treatment is often associated with difficulties which may arise out of two reasons. The first is the need to identify the genus or even better the particular strain to be treated, in order to select the suitable antibiotic. Another difficulty relies in the resistance of Enterococci to particular antibiotics. Due to vertical and horizontal gene transfer bacteria may “collect” resistance determinants from other organisms rendering them more unsusceptible on the one hand to different antibiotics and on the other to particular concentrations of an antibiotic, which may have proven in an earlier treatment efficient with the same or even lower doses.

Particularly during the last decade, vancomycin-resistant Enterococci (VRE) have emerged as important causes of nosocomial infections. VRE are often resistant to a variety of antibiotics, seriously constraining treatment of infections (Cetinkaya, Y. et al.; Clin. Microbiol. Rev.; 2000, 13:686-707). The species responsible for most infections are Enterococcus (E.) faecalis and E. faecium. E. faecium, which is intrinsically more resistant than E. faecalis, accounts for approximately 10% of enterococcal infections overall, but in recent years for a disproportionate number of nosocomial infections. Particularly, the frequent horizontal acquisition of resistance traits by this species has resulted in nosocomial E. faecium infections exceedingly difficult to treat. The emergence of Enterococci resistant to most or all licensed antibiotics leaves few treatment options and recent studies have shown that 36.6% of those patients with VRE in blood died as compared with 16.4% of those with vancomycin sensitive Enterococci.

Up to now, detection of Enterococci has been performed by isolating nucleic acid sequences from clinical samples and analyzing them by either using gel electrophoresis of DNA fragments (e.g. of restriction fragments), hybridization events, and the direct sequencing of DNA (for example according to the Maxam-Gilbert method). All of the above-mentioned methods are commonly used in biological sciences, medicine and agriculture. The deficiencies of the above methods reside, however, in that even though southern blots and hybridization experiments may be carried out relatively fast, they are only useful for the analysis of short DNA strands. The DNA sequencing results in the accurate determination of the nucleic acid sequences, but is time consuming, expensive and connected with certain efforts when applied to greater projects, e.g. the sequencing of a complete genome.

Another possibility, which requires at least less efforts, is disclosed in US 2002/132,285 and US 2004/241,747. In said documents, a detection medium for van A and van B vancomycin resistant Enterococci is described, which allows the selective growth of vancomycin resistant strains, so that both the identification of the strain and the resistant determinant is possible. Such a medium bases on specific nutrient indicators, which only the target microbe can significantly metabolize and use for growth. Since these phenotypic based microbiological and biochemical techniques for species identification and antibiotic susceptibility determination require at least two days, a reliable therapy is not possible in urgent cases of critical ill patients.

Other methods for the detection of vancomycin-resistant Enterococci are based on real-time PCR. E.g. in US 2004/058336 respective primers and other constituents of a kit allowing the selective detection of Enterococci is described. Similar means are provided in U.S. Pat. No. 6,054,269, wherein polynucleotides and oligonucleotides, useful as both probes and primers, for an identification of species of the Streptococcus genus and the Enterococcus genus are disclosed. Polypeptides expressed by the polynucleotides and oligonucleotides may also utilized for the preparation of monoclonal and polyclonal antibodies that recognize the polypeptides. In addition, antibody-basing tests have been performed.

The micro-array technology represents in contrast to the above mentioned methods, a tool for a highly specific, parallel detection of numerous different DNA sequences in a single experiment. Micro-arrays which are in some cases also referred to as hybridization arrays, gene arrays or gene chips comprise in brief a carrier or support on which at defined locations at a possibly high density capture molecules are attached directly or via a suitable spacer molecule. The spacer molecules may be considered to function as a “bridge” between the capture molecule and the surface of the carrier to allow an easier attachment of the capture molecule. Said capture molecules consist of relatively short nucleic acid sequences, in particular DNA, which is capable to hybridize specific to the target molecules or probe molecules to be analyzed resulting usually in DNA:DNA or DNA:RNA hybrids. The occurrence of the hybridization event is than detected with for example fluorescent dyes and analyzed.

The advantages of the micro-array concept resides preliminary in its ability to carry out very large numbers of hybridization-based analyses simultaneously. Originally developed for the analysis of mammalian gene expression, an increasing number of reports on micro-arrays for identification and characterization of prokaryotes also used in microbial diagnostics was encountered in recent years (Bodrossy, L. and A. Sessitsch; Curr. Opin. Microbiol. 7 (2004), 245-254). Combination of PCR based pre-amplification steps with subsequent micro-array based detection of amplicons on a micro-array facilitates the sensitive and highly specific detection of PCR products (Call, D. R. et al.; Int. J. Food Microbiol. 67 (2001), 71-80). Amplicons are identified by a specific hybridization reaction on the array thus reducing the risk of wrong positive results due to the occurrence of nonspecific bands after PCR. Besides that, micro-arrays utilizing oligonucleotides as capture probes enable the detection of single nucleotide polymorphisms (SNPs) such as resistance mutations without the need for additional sequencing. However, only a few studies describe the development of diagnostic micro-arrays for the molecular detection of bacterial antibiotic resistance, targeting either a limited number of acquired antibiotic resistance genes or resistance mutations in various genes.

The use of micro-arrays for the detection of pathogenic bacteria is for example disclosed in WO 03/031654, wherein a micro-array with probes for genotyping Mycobacteria species, differentiating Mycobacterium strains and detecting antibiotic-resistant strains is specified. Methods for assaying drug resistance and kits for performing such assays are disclosed in the U.S. Pat. No. 6,013,435. Target sequences associated with genetic elements are selectively amplified and detected. The methods described herein are especially useful for screening of Microorganisms, which are difficult to culture. In US-2003143591 methods and strategies to detect and/or quantify nucleic acid analytes in micro-array applications such as genotyping (SNP analysis) are disclosed. Nucleic acid probes with covalently conjugated dyes are attached either to adjacent nucleotides or at the same nucleotide of the probe while novel linker molecules attach the dyes to the probes.

Disadvantages of the methods and techniques according to the state of the art for the detection of bacteria of the genus Enterococcus reside in that they require long runs and are solely adaptive to a limited number of samples to be tested and often also expensive. Additionally, no method is known which is capable to clearly identify the presence of Enterococci and uses moreover simultaneously several nucleic acids probes for the detection of multiple antibiotic resistance genes and optionally other virulence factors to facilitate an overview on the resistance properties and gives fast valuable and sometimes life-saving information about a suitable treatment. Another problem in dealing with the analysis of clinical material or probes is its unpredictability. One is never sure at which point of time the sample will be available and the condition in which it will arrive. Considering the number of steps and personnel involved in moving a specimen from the patient to the laboratory and then its analysis, there is also high need to standardize the diagnostic protocol especially to control the storage and extraction of the sample and to include adequate controls that can be used to validate data on the clinical sample.

The present invention aspires to overcome the problem associated with prior art methods.

### SUMMARY OF THE INVENTION

The present invention provides a micro-array as a genotype based method, which allows both determination of the presence of Enterococci in a sample and likewise detection of antibiotic susceptibility of bacteria of the genus Enterococcus. The micro-array incorporates on the one hand nucleic acids allowing the identification and on the other nucleic acids for targeting resistance genes of eventually multi-resistant Enterococci. The micro-array enables a rapid, accurate and inexpensive identification of antibiotic resistance profiles of bacteria of the genus Enterococcus in a standardized manner.

Nucleic acids characterizing the most predominant Enterococci associated with nosocomial outbreaks, like the enterococcal surface protein (esp), phoshoribosylamino-imiazolcarboxylase ATPase subunit (purK) gene and hyaluronidase (hyl) gene, and allowing by identification of polymorphisms the distinction between E. faecium and E. faecalis, such as D-alanine:D-alamine ligase (ddl) gene, may bc included as well, which genes broaden the information about the virulence potential and permits at the same time an overview about the enterococcal bacteria contained, preliminary information about the presence of E. faecium and E. faecalis. The micro-array is easily expandable and may thus be adapted to changing clinical and epidemiological requirements in clinical diagnosis as well as in epidemiological studies. A fast and reliable assay with a high throughput may be helpful in reducing the spread of multi-resistant isolates and improves the treatment options of severe and often life-threatening Enterococci infections. The present microarray may also help to update the understanding of the prevalence of different forms of MLS resistance and to compare the in-vitro/in-vivo activities of MLS antibiotics.

### BRIEF DESCRIPTION OF THE DRAWINGS

FIG. 1 shows the single and multiplex PCR amplifications for PCR1 and PCR2.

FIG. 2 illustrates the nucleotide distribution at specific loci for five different purK alleles. The arrow indicates the most relevant position no. 115 distinguishing the five allele types from all others described so far. Nucleotide determination at the four other loci facilitates final discrimination between the five respective allele types.

FIG. 3 shows an example of an array layout.

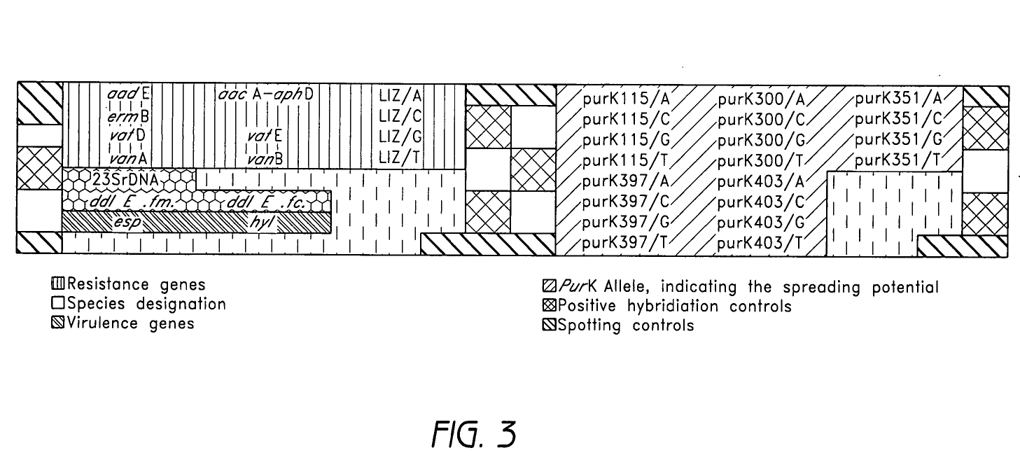

FIG. 4 displays results of microarray hybridizations with fluorescently labeled multiplex PCR products derived from two enterococcal isolates E. faecium UW 5911 and E. faecalis UW 6124. The respective genotypes of the strains are indicated.

### DETAILED DESCRIPTION OF THE PREFERRED EMBODIMENT

The term “micro-array” as used herein refers to a carrier or support respectively, which is preferably solid and has a plurality of molecules bound to its surface at defined locations or localized areas. The molecules bound to the carrier comprise nucleic acid sequences, the capture molecules, which are specific for a given or desired target sequence. The sequences may be bound to the carrier via spacer molecules, which bind each capture nucleotide to the surface of the support. In the above context a localized area is an area of the carrier's surface, which contains capture molecules, preferably attached by means of spacers to the surface of the carrier, and which capture molecules are specific for a determined target/probe molecule.

“Spacers” are molecules that are characterized in that they have a first end attached to the biological material and a second end attached to the solid carrier. Thus, the spacer molecule separates the solid carrier and the biological material, but is attached to both. The spacers may be synthesized directly on or may be attached as a whole to the solid carrier at the specific locations, whereby masks may be used at each step of the process. The synthesis comprises the addition of a new nucleotide on an elongating nucleic acid in order to obtain a desired sequence at a desired location by for example photolithographic technologies which are well known to the skilled person. Bindings within the spacer may include carbon-carbon single bonds, carbon-carbon double bonds, carbon-nitrogen single bonds, or carbon-oxygen single bonds. The spacer may be also designed to minimize template independent noise, which is the result of signal detection independent (in the absence) of the template. In addition, the spacer may have side chains or other substitutions. The active group may be reacted by suitable means to form for example preferably a covalent bound between the spacer and solid carrier, capture or probe molecule. Suitable means comprise for example light. The reactive group may be optionally masked/protected initially by protecting groups. Among a wide variety of protecting groups, which are useful are for example FMOC, BOC, t-butyl esters, t-butyl ethers. The reactive group is used to build to attach specifically thereto (after the cleavage of the protecting group) another molecule.

The “localized area” is either known/defined by the construction of the micro-array or is defined during or after the detection and results in a specific pattern. A spot is the area where specific target molecules are fixed on their capture molecules and approved by a detector.

As used herein, the term “carrier” or “support” refers to any material that provides a solid or semi-solid structure and a surface allowing attachment of molecules. Such materials are preferably solid and include for example metal, glass, plastic, silicon, and ceramics as well as textured and porous materials. They may also include soft materials for example gels, rubbers, polymers, and other non-rigid materials. Preferred solid carriers are nylon membranes, epoxy-glass and borofluorate-glass. Solid carriers need not be flat and may include any type of shape including spherical shapes (e.g., beads or microspheres). Preferably solid carriers have a flat surface as for example in slides (such as object slides) and micro-titer plates, wherein a micro-titre plate is a dished container having at least two wells.

The expression “attached” describes a non-random chemical or physical interaction by which a connection between two molecules is obtained. The attachment may be obtained by means of a covalent bond. However, the attachments need not be covalent or permanent. Other kinds of attachment include for example the formation of metalorganic and ionic bonds, binding based on van der Waal's forces, or any kind of enzyme substrate interactions or the so called affinity binding. An attachment to the surface of a carrier or carrier may be also referred to as immobilization.

A “target gene” or “resistance gene” relates to a factor responsible for the development of resistance in Enterococci, which may be acquired by the micro-organism via horizontal and also vertical gene transfer and which actively counteracts the effect of an antibiotic by different modes of action. Particularly, genes conveying resistance to antibiotics, such as the aminoglycoside adenyltransferase (aadE) gene, bifunctional aminoglycoside modifying enzyme (aacA/aphD) gene, erythromycin ribosome methylase (ermB) gene, streptogramin A acetyltransferase (vatD) gene, streptogramin A acetyltransferase (vatE) gene, vancomycin resistance protein A (vanA) gene and vancomycin resistance protein A (vanA) gene, which may be normally present on plasmid(s) or also may be incorporated in the genome of Enterococci, are envisaged.

The terms “complementary” or “complementarity” are used in reference to polynucleotides (i.e., a sequence of nucleotides such as an oligonucleotide or a target nucleic acid) in the light of the base-pairing rules. Complementarity may be partial, in which only some bases of the nucleic acids are matched according to the base pairing rules. Alternatively, there may be a complete complementarity between the nucleic acids in such a way that there are no mismatches. The degree of complementarity between nucleic acid strands has significant effects on the stringency and strength of the hybridization between two different nucleic acid strands. Complementarity as used herein is not limited to the predominant natural base pairs. Rather, the term also encompasses alternative, modified and non-natural bases, including but not limited to those that pair with modified or alternative patterns of hydrogen. With regard to complementarity, it is important for some applications to determine whether the hybridization represents a complete or partial complementarity. If it is desired for example to detect the presence or absence of a particular DNA (such as from a virus, bacterium, fungi or protozoan), the only important condition is that the hybridization method ensures hybridization when the relevant sequence is present. Other applications in contrast, may require that the hybridization method distinguish between partial and complete complementarity, for example in the detection of genetic polymorphisms.

The term “homology” and “homologous” refers to a degree of identity. There may be partial homology or complete homology. A partially homologous sequence is one that is less than 100% identical to another sequence.

“Hybridization” is used in reference to the pairing of complementary nucleic acids. Hybridization and the strength of hybridization (i.e., the strength of the association between the nucleic acids) is influenced by such factors as the degree of complementarity between the nucleic acids, stringency of the conditions involved, and the melting temperature of the formed hybrid. Hybridization involves the annealing of one nucleic acid to another complementary nucleic acid, i.e., a nucleic acid having a complementary nucleotide sequence.

“Stringency” refers to the conditions, which are involved in a correct hybridization event, for example temperature, ionic strength, pH and the presence of other compounds, under which nucleic acid hybridizations are conducted. Under conditions of high stringency, nucleic acid base pairing will occur only between nucleic acid fragments that have a high frequency of complementary base sequences. Thus, conditions of weak or low stringency are often required when it is desired that nucleic acids that are not completely complementary to one another be hybridized or annealed together.

A “marker” or “label” refers to any atom or molecule that may be used to provide a detectable (preferably quantifiable) effect and that can be attached to a nucleic acid. Markers may include colored dyes; radioactive labels; binding moieties such as biotin; haptens such as digoxgenin; luminogenic, phosphorescent or fluorogenic moieties; and fluorescent dyes alone or in combination with moieties that can suppress or shift emission spectra by the energy transfer of fluorescence. Markers may provide signals, which are detectable for example by fluorescence, radioactivity, colorimetry, gravimetry, X-ray diffraction or absorption, magnetism and enzymatic activity. A marker may be a charged moiety (positive or negative charge) or may also have a neutral charge. They may include or consist of nucleic acid or protein sequence. Preferred markers are fluorescent dyes.

A “target” or “probe molecule” refers to a nucleic acid molecule to be detected. Target nucleic acids may contain a sequence that has at least a partial complementarity with at least a probe oligonucleotide.

“Probes” or “probe molecules” refer to nucleic acids, which interact with/hybridize to a target nucleic acid to form a detection complex.

The term “signal probe” or “probe” relates to a probe molecule, which contains a detectable moiety, which are already outlined above.

The term “nucleic acid” is intended to comprise any sequence of deoxyribonucleotides, ribonucleotides, peptido-nucleotides, including natural and/or artificial nucleotides.

The expression “sample” is meant to include any specimen or culture of biological and environmental samples or nucleic acid isolated therefrom. Biological samples may be animal, including human, fluid, such as blood or urine, solid or tissue, alternatively food and feed products and ingredients such as dairy items, vegetables, meat and meat by-products. Environmental samples include environmental material such as surface matter, soil, water, industrial samples and waste, for example samples obtained from sewage plant, as well as samples obtained from food and dairy processing instruments, apparatus, equipment, utensils, disposable and non-disposable items. The sample may be used as such in the assay or may be subjected to a preliminary selection step, such as e.g. culturing the sample under conditions favoring or selecting for Enterococci in said sample. Also, the nucleic acids contained in the sample may be isolated prior to performing the assay. In the presence of a multi-resistant Enterococci in the sample the resulting nucleic acid sample will contain the target nucleic acid which may be isolated from the biological sample in any way known to the skilled person, including conventional isolation comprising lysis of the cellular material of the biological sample and isolation of DNA or RNA therefrom. In case the target nucleic acid is present in a low amount, the said nucleic acid may be subjected to PCR, preferably to a multiplex PCR, to specifically amplify the target nucleic acid prior to performing the assay.

A “nucleic acid sample” may be a polynucleotide or oligonucleotide of a variable length and is represented by a molecule comprising at least 5 or more deoxyribonucleotides, preferably about 10 to 1000 nucleotides, more preferably about 20 to 800 nucleotides and more preferably about 20 to 100 or even more preferred about 20 to 60. The exact size will depend on many factors, which in turn depend on the ultimate function or use of the oligonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another.

According to an embodiment, the present micro-array comprises a carrier or support on which in the form of a specific pattern nucleic acids are immobilized. Said nucleic acids comprise sequences specific for at least 4 target genes of Enterococci and at least one gene characteristic for bacteria of the genus Enterococcus. For a correct determination of the presence of multi-resistant Enterococci in a sample a number of at least four target genes have proven to yield a doubtless, non-ambiguous result, even if for a better overview about the resistance profile more than four target genes may be incorporated.

Said immobilized nucleic acids comprise sequences specific for at least 4 target genes of Enterococci, which sequences are preferably randomly selected from the group consisting of aadE, aacA-aphD, ermb, vat(D), vat(E), vanA and vanB. Each of these target genes is detected either by a single capture probe. For a correct and unambiguous identification of the strain and the detection of a multi-resistant Enterococci strain 4 target genes, which include resistance genes, have proven to be sufficient without any requirements concerning the selection of the target genes. The detection of 5 or more target genes is preferred, since in this case more precise information about antibiotic determinants are achieved and a possible therapy is eased. The present micro-array may also comprise nucleic acids probes specific for at least 6 target genes and more preferably 7 target genes.

The at least one gene characteristic for bacteria of the genus Enterococcus is preferably 23S rDNA. In case said gene is used, it has to be characterized by four nucleic acid sequences, required to provide a detectable hybridisation event under stringent conditions. In consequence, also four nucleic acid capture probes corresponding to known single nucleotide polymorphisms (SNPs) are attached to the surface of the carrier of the present micro-array to act as the capture molecule for the 23S rDNA, thereby allowing the individual and unambiguous detection of each SNP. The four different capture probes (for the different SNPs) for the gene may be attached to the carrier (e.g. spotted) on one localized area or on different ones.

The inclusion of Enterococci specific control capture probes (preferably hyl, esp, ddl and purK) as well as capture probes for the detection of the presence of other organisms (preferably a Arabidopsis thaliana gene and S. aureus gene) allows a more correct species identification. The hyaluronidase (hyl) gene is indicative for E. faecium only, whereas enterococcal surface protein (esp) gene and the phoshoribosylaminoimiazolcarboxylase ATPase subunit (purK) gene denote the presence of particularly virulent enterococci sometimes referred to as C1 population of E. faecium (Homan, W. L. et al.; J. Clin. Microbiol.; 2002, 40:1963-1971). The D-alanine:D-alanine ligase (ddl) gene allows to distinguish between E. faecium and E. faecalis. The last two genes, purK and ddl, may be also characterized by more than one nucleic acid, in order to cover the full spectrum of SNPs.

Due to difficulties that occurred when deducing primers with similar melting temperatures to allow consistent hybridisation results, it is surprising that in fact all of the above mentioned nucleic acid sequences deduced from the target genes, the gene characteristic for bacteria of the genus Enterococcus and the control probes may be used in one single micro-array and result nevertheless in high-reliable results. The length of the sequence specific for the respective target gene is about 15 to 500, preferably 15 to 300, more preferably 15 to 100, even more preferred 15 to 70, even more preferred, 15 to 50, or 20 to 40.

The carrier or support of the present DNA micro-array may consist of different materials, preferably of glass, silicon, silica, metal, plastics or mixtures thereof prepared in format selected from the group of slides, discs, gel layers and/or beads. The carrier may also be a micro plate or a slide and may consist of epoxy glass. A preferred support is an epoxy modified glass slide (Elipsa AG, Berlin, Germany).

Preferably, the present micro-array has at least 100 molecules attached per square centimeter of the solid carrier. This density may be, however, higher and be adapted to the respective application of the micro-array, in that also other suitable applications may be performed, e.g. for the determination of resistances in other organisms different from Enterococci and/or for the detection of resistance gene(s), which are unknown yet to play a role in Enterococci. For example, the density of the nucleic acids probes attached per square centimeter of solid carrier amounts more preferably at least to 1.000, still more preferably at least to 5.000 and most preferably at least to 10.000 nucleotides per square centimeter.

Said specific pattern allows the mapping of each nucleic acid probe to a specific position on said carrier and a specific analysis, in that the analysis of the results of the present micro-array is facilitated and non-ambiguous concerning the attribution of a particular spot to a previous attached nucleic acid probe.

Spacer molecules of any length may be arranged between the carrier and the nucleic acids applied on the carrier. The spacer may be for example polymer-based spacers, but may also consist of an alkane chain, or any derivatives thereof, of a suitable length, which comprises at each end respective functional groups for attachment to the solid support and the nucleic acid probe. Preferably, 15-thymidine spacers have been attached with one end to the surface of the support and with the other end to the 3′-terminal end of the respective nucleic acid to be immobilized.

According to another preferred embodiment, the present invention provides a method for the detection of multi-resistant Enterococci strains in a sample material, using a micro-array for the detection of target genes conferring antibiotic resistance and at least one gene characteristic for bacteria of the genus Enterococcus.

The method comprises the step to obtain a sample material of interest. Prior to performing the method of the present invention the sample may be pre-treated e.g. centrifuging or filtering to separate non-soluble matter or selecting for Enterococci in the sample. This may be achieved by e.g. culturing the sample under conditions favouring the growth of Enterococci. Also, to improve performance, nucleic acids contained in the sample material may be isolated and/or amplified by using standard techniques. The sample and/or the isolated/purified nucleic acid material is applied to the surface of the present micro-array. Said sample is now allowed to hybridize to the immobilized nucleic acids, the capture probes, for targeting at least 4 target genes of Enterococci and at least one gene characteristic for bacteria of the genus Enterococcus. By choosing suitable hybridisation conditions known to the skilled person, such as e.g. applying a certain stringency during hybridization and washing (cf. Sambrook et al., Molecular Cloning—A Laboratory Manual, Third Edition, Cold Spring Harbor, 2001), only those nucleic acids will hybridize to the immobilized nucleic acids and/or remain bound during washing steps, which exhibit a high homology to the immobilized nucleic acids.

Said nucleic acids probes specific for targeting at least 4 target genes of Enterococci are preferably randomly selected from the group consisting of aadE, aacA-aphD, ermB, vat(D), vat(E), vanA and vanB. Each of these target genes is detected by a specific capture probes, For a correct and non-ambiguous identification of the strain and the determination of a multi-resistant Enterococci strain 4 target genes and one gene characteristic for bacteria of the genus Enterococcus have proven to be sufficient without any requirements concerning the selection of the target genes. Preferably, the micro-array may also comprise nucleic acids specific for at least 5 target genes, more preferably at least 6 target genes and most preferably 7 target genes.

The nucleic acid probe specific for at least one gene characteristic for bacteria of the genus Enterococcus is preferably 23S rDNA. In case said gene is used, it has to be characterized by four nucleic acid probes according to the four SNPs said gene embraces.

Enterococci specific control probes (preferably hyl, esp, ddl and purK) may be included. Other controls are probes, which are capable to detect the presence/absence of other organisms (preferably a Arabidopsis thaliana and S. aureus gene) and may be also included for a correct species identification.

The nucleic acid sample to be used for hybridizing to the immobilized nucleic acids consists preferably of oligonucleotides and/or polynucleotides of a length between 10 and 1000 nucleotides each, preferably shorter oligonucleotides/polynucleotides exhibiting a length of about 10 to 100 or between 20 to 60. The length may be obtained for example by the digestion of plasmid or genomic DNA with DNAse or preferably restrictions enzymes and facilitates the hybridisation.

The nucleic acid sample, which comprises oligonucleotides and/or polynucleotides, is preferably isolated from body tissues or fluids, particularly blood, suspected to contain Enterococci, followed by the isolation and optional the amplification of the DNA and/or RNA contained therein by PCR techniques, such as a multiplex PCR, which allows the amplification of several DNA fragments in one PCR reaction. Such techniques are well known to the skilled person and may be also performed with commercial available kits.

The capture and the target nucleic acids may be present in a labeled form. The target nucleic acids may be labeled prior to performing the assay, by including a marker molecule into the molecule, e.g. during its amplification or isolation. Said marker molecule is preferably a fluorescent marker. Also the capture molecules may be labeled, in case of a fluorescent dye preferably with a dye exhibiting a different excitation and/or emittance wavelength, which allows a normalization of the experiment.

Methods for the detection of binding include e.g. surface plasmon resonance or detection of fluorescence at a localized area indicative of binding of a labelled molecule. Fluorescence may be detected e.g. via confocal laser induced fluorescence.

In another embodiment of the invention, a diagnostic kit is provided for the detection of Enterococci infections.

Said kit either provides the nucleic acids specific for 7 target genes of Enterococci, which are selected from the group consisting of aadE, aacA-aphD, ermB, vat(D), vat(E), vanA and vanB, and nucleic acids specific for at least one gene characteristic for bacteria of the genus Enterococcus or a micro-array as described above. The kit may also contain respective means, such as buffers, chemicals, manual, to assist the purchaser in the detection of multi-resistant Enterococci.

Preferably four nucleic acids specific for 23S rDNA is included, which gene is characteristic for bacteria of the genus Enterococcus.

Additionally, the kit may also include the appropriate controls, in that probes are included which are preferably specific for the hyl, esp, ddl and purK genes and a Arabidopsis thaliana and S. aureus gene.

A typical automated processing of a micro-array according to a preferred embodiment of the present invention includes the use of three components. First, the micro-array or support respectively, second a reader unit and third means for the evaluation of the results, e.g. a suitable computer software. The reader unit comprises in general a movable tray, focussing lens(es), mirrors and a suitable detector, e.g. a CCD camera. The moveable tray carries the micro-array and may be moved to place the micro-array within the light path of one or more suitable light sources, e.g. a laser with an appropriate wavelength to excite a fluorescent compound. The evaluation program or software may serve for example to recognize specific patterns on the array or to analyse different expression profiles of genes. In this case, the software searches colored points on the array and compares the intensity of different color spectra of the same point. The result may be interpreted by an analyzing unit and afterwards stored in a suitable file format for further processing.

As detailed above, the probe- and/or target-nucleic acids may be labelled each with a fluorescent dye and the intensity of the fluorescence at different wavelengths of each point is compared to the background. The detector, e.g. a photomultiplier or CCD array, transforms low light intensities to an amplifiable electrical signal. Other methods use different enzymes, which are covalently bound to the nucleotide by means of a linker molecule. The enzymatic colorimetry uses for example alkaline phosphatase and horseradish peroxidase as marker. By contacting with a suitable molecule, a detectable dye may be achieved. Other chemoluminescent or fluorescent marker comprise proteins capable to emit a chemoluminescent or fluorescent signal, if irradiated with light of a discrete, specific wavelength, e.g. 488 nm for the green fluorescent protein. Radioactive markers are applied in case of low detection limits are required, but are due to their harmful properties not wide spread. Fluorescence marking is performed with nucleotides linked to a fluorescent chromophore. Combinations of nucleotides and fluorescent chromophore comprise in general Cy3 (cyanine 3)/Cy5 (cyanine 5) labelled dUTP as dye, since they may be easily incorporated, the electron migration for fluorescence may be exited by means of customary lasers and they also have distinct emission spectra.

The hybridisation of micro-arrays essentially follows the conventional conditions of southern or northern hybridisations, which are well known to the skilled person. The steps comprise a pre-hybridisation, the intrinsic hybridisation and a washing step after hybridisation occurred. The conditions have to be chosen in such a way that background signals are kept low, minimal cross-hybridisation (in general a reduced number of mismatches) occurs and with a sufficient signal strength, which has to be proportional for some applications to the concentration of the target molecule.

The hybridisation event may be detected generally by two different kinds of array-scanners. One method employs the principle of the confocal laser microscopy, which uses at least one laser to scan the array in point-to-point manner. Fluorescence is than detected by photomultipliers, which amplify the emitted light. The cheaper GGD basing readers use typically filtered white light for the excitation. The surface of the array is scanned with this method in sections, which allows the faster achievement of results of a lower significance.

Also, the so-called gridding for the analysis of the results, in which an idealised model of the layout of the micro-array is compared with the scanned data to facilitate the spot definition. Pixels are classified (segmented) as spot (foreground) or background to produce the spotting mask. Segmentation techniques may be divided in fixed segmentation circle, adaptive circle segmentation, adaptive shape segmentation and histogram segmentation. The use of these techniques depends from the shape of the spots (regular, irregular) and the quality of the proximal arrangement of the spots.

Another issue is the intensity of the distinct spots, since the concentration of hybridised nucleotides in one spot is proportional to the total fluorescence of this spot. In particular, the overall pixel intensity and the ratio of the different fluorescent chromophores used (in case of Cy3 and Cy5, green and red) are important for the calculation of the spot intensity. Beneath the spot intensity, also the background intensity has to be taken into account, since various effects may disturb the fluorescence of the spots, for example the fluorescence of the support and of the chemicals used for the hybridisation. This may be performed by the so-called normalisation, which includes the above-mentioned effects and others like fluctuations of the light source, the lower availability/incorporation of the distinct marker molecules (Cy5 worse than Cy3) and their differences in emission intensities. Of importance for the normalisation is further the reference against which shall be normalized. In general, this may be a specific set of genes or a group of control molecules present on the micro-array.

The results may be further processed by means of the available software tools and according to the knowledge of bioinformatics.

The present invention provides a method, a micro-array and kit for the detection of enterococcal infections, helpful in reducing the spread of multi-resistant isolates and improve the treatment options of severe and sometimes life-threatening enterococcal infections. The kit allows advantageously the detection of the prevalence and dissemination of multi-resistant bacteria belonging to the genus Enterococcus. The present micro-array for the detection of enterococcal infections has also shown a surprising high coincidence with results from phenotypic resistance testing and PCR based detection methods.

It is to be understood, that the above description is intended to be illustrative and not restrictive. Many embodiments will be apparent to those of skilled in the art upon reviewing the above description. By way of example, the invention has been described preliminary with reference to the use of nucleic acids comprising sequences specific for the target genes of Enterococci and for said at least one gene characteristic for bacteria of the genus Enterococcus. It should be clear that also other target genes but also virulence factors may be selected in dependence from the genetic development of multi-resistant Enterococci strains and may be consequently characterised by more than one nucleic acid sequence according to the number of SNPs developed. The scope of the invention should, therefore, be determined not with reference to the above description, but should instead be determined with reference to the appended claims, along with the full scope of equivalents to which such claims are entitled.

### EXAMPLES

Bacterial Strains and DMA Extraction

Enterococcal isolates investigated in this study originated from material sent to our reference laboratory. To evaluate oligonucleotide capture probes for the detection of various resistance and virulence genes, the following, previously characterized strains were used: E. faecium UW 1965 (reference strain for aacE, ermB, vafE), E. faecalis UW700 (reference strain for aacA-aphD, vanB), E. faecium UW1342 (reference strain for vanA, vatD), E. faecium UW 5248 (reference strain for purK20, esp, hyl), E. faecalis UW 5245 (reference strain for esp), E. faecium UW5256 (reference strain for purK1, esp, hyl). All strains were grown on sheep blood agar. Genomic DNA was extracted from 2 ml overnight culture with the DNeasy Tissue Kit (Qiagen, Hilden, Germany) following the manufacturer's instructions.

Antimicrobial Susceptibility Testing

All isolates were tested with the broth microdilution assay as described in the NCCLS standard (Grimm, V et al.; J. Clin. Microbiol. 2000, 42:3766-3774), except that Iso-Sensitest broth (Oxoid, Wesel, Germany) was used.

Primers and Probes

The primers used to amplify 13 different loci in two multiplex PCR reactions are shown in Table 1; all capture probes used in the study are in Table 2. Primers and probes were selected from public databases using the software Primer3 freely available via the internet (http://frodo.wi.mit.edu/cgi-bin/primer3/primer3_www.cgi), and synthesized by Metabion (München, Gemany). Oligonucleotide capture probes were synthesized with a 5′-terminal amino-modification for covalent coupling to the slide surface and a 10 residues T pacer to improve hybridization efficiency. All probes were designed with similar melting temperatures (Table 2) to facilitate uniform hybridization conditions and to prevent high divergence in signal intensities. Specificity of the probes was verified in a BLAST search available through the National Center for Biotechnology Information website (wwwincbi.nlm.nih.gov).

Controls

In addition to the amplicon specific capture probes several control probes were designed, as described previously. Briefly, a fluorescein labeled spotting control (NH2-mecA-F) was used to check the spotting quality and to facilitate orientation on the array; negative and positive hybridization controls (NegHybProbe and HybProbe, respectively) were selected to control the hybridization step; the latter one was complementary to a fluorescein labeled oligonucleotide (HybTarget), which was spiked during the hybridization step; process controls (Table 2), targeting the PCR amplification controls, monitored the efficiency of PCR amplification, labeling and hybridization.

TABLE 1

| | | | | | |
|---|---|---|---|---|---|
| | | | Product | GeneBank | |
| | | | length | accession no./ | |
| Target gene | Primer | Sequence (5′→3′) | (bp) | reference | PCR |

| | | | | | |
|---|---|---|---|---|---|
| aadE | aadE1 | GCA GAA CAG GAT GAA CGT ATT CG | 369 | AF330699 | |
| | aadE2 | ATC AGT CGG AAC TAT GTC CC | | | |
| aacA-aphD | aacA-aphD 1 | TAA TCC AAG AGC AAT AAG GGC | 227 | M18086 | |
| | aacA-aphD 2 | GCC ACA CTA TCA TAA CCA CTA | | | |
| ermB | ermB1 | TTT TGA AAG CCG TGC GTC TG | 202 | AF229200 | |
| | ermB2 | CTG TGG TAT GGC GGG TAA GTT | | | |
| vat(D) | vatD1 | CAT AGA ATG GAT GGC TCA AC | 166 | AF368302 | |
| | vatD2 | CAT CCC CGA TTT TTA CTC CT | | | Multiplex |
| | | | | | PCR I |
| vat(E) | vatE1 | CTATAC CTG ACG CAA ATG C | 511 | AF139725 | |
| | vatE2 | GGT TCA AAT CTT GGT CCG | | | |
| vanA | vanAB1 | GTA GGC TGC GAT ATT CAA AGC | 230 | M97297 | |
| | vanA2 | CGA TTC AAT TGC GTA GTC CAA | | | |
| vanB | vanAB1 | GTA GGC TGC GAT ATT CAA AGC | 330 | U00456 | |
| | vanB2 | GCC GAC AAT CAA ATC ATC CTC | | | |
| 23S rDNA^{a} | rDNA1 | AGA GTT TGA TCC TGG CTC AG | ˜1500 | AF515223(6) | |
| (ubiquitous | rDNA2 | AAG GAG GTG ATC CAR CCG CA | | | |
| enterococcus) | | | | | |
| hyl^{b} | hyl1 | GAA ATG CGC CTC TCT CTT TTT | 162 | AF544400.1 | |
| | hyl2 | GCT AGC CTC AGC AGC AGA TAA | | | |
| esp | espTIM1 | CTT TGA TTC TTG GTT GTC GGA TAC | 475 | AJ487981 | |
| | espTIM2 | TCC AAC TAC CAC GGT TTG TTT ATC | | | |
| ddl | ddl1 | GGA GGA CAA KCW TTT GAA GAT TA | ˜535 | U00457, | Multiplex |
| | ddl2 | CGG ATA AAK YAA AGA ACC TTC AC | | AF550665 | PCR II |
| purK^{b} | purK1 | GCA GAT TGG CAC ATT GAA AGT | ˜650 | :www.mlst.net] | |
| | purK2 | TAC ATA AAT CCC GCC TGT TTY | | | |
| 23S rDNA | 23S LIZ1 | TGG GCA CTG TCT CAA CGA | 633/634 | EFA295305(12) | |
| linezolid | 23S LIZ2 | GGA TAG GGA CCG AAC TGT CTC | | | |
| resistance | | | | | |
| determinating | | | | | |
| region^{a} | | | | | |
| (ubiquitous | | | | | |
| enterococcus) | | | | | |

^{a}PCR amplification control
^{b}E. faecium only

TABLE 2

| Target gene | | | T_{M}^{a} | |
|---|---|---|---|---|
| or mutation | Capture Probe | Sequence (5′→3′) | (° C.) | Comment |
| aadE | NH₂-aadE | TAT TCC CAA ATT GAT TAA GCC AGT | 58 | |
| aacA-aphD | NH₂-aacA-aphD | GAA CAT GAA TTA CAC GAG GGC AAA | 62 | |
| ermB | NH₂-ermB | TCG GTG AAT ATC CAA GGT AC | 56 | |
| vat(D) | NH₂-vatD | TCC TGG CAT AAT TAC AAC ATC TT | 58 | |
| vat(E) | NH₂-vatE | CAT TAT CGG AGC AAA TAG TG | 54 | PCR amplicon |
| | | | | specific capture |
| | | | | probes |
| vanA | NH₂-vanA | GCT ATT GAC TTT TTT CAC ACC G | 58 | |
| vanB | NH₂-vanB | TGG CGT AAC CAA AGT AAA CAG T | 58 | |
| 23S rDNA^{b} | NH₂-Edffhp201 | ATC AGC GAC ACC CGA AAG | 56 | |
| hyl^{d} | NH₂-hyl | CAT CGT AGA GTT CAC GCC ATT | 60 | |
| esp | NH₂-esp | ACC TGT TCC ATA AGT RTT CTG RA | 61 | |
| ddl | NH₂-ddl_E_fm | GTG GAC AGA CAG AGG AAG G | 60 | species specific |
| | NH₂-ddl_E_fc | TCG CCT GTT TCT TCA GGT G | 57 | detection of the |
| | | | | ddl amplicon |
| purK^{c,d} | NH₂-purK115 | ATC ACG AAG TTA(C/G/T) TTC GAT TCC AA | 58-60 | purK allele |
| | NH₂-purK300 | GAA GGA ACC TA(C/G/T)T GTT TTA GAA | 53-55 | determination |
| | NH₂-purK351 | CCA TTT CCT A(C/G/T)CC ACC TTG AT | 56-58 | |
| | NH₂-purK397 | TGG TGG ATA(C/G/T) TTT TCC TCG ACC | 60-62 | |
| | NH₂-purK403 | ATA TTG TTG TGA(C/G/T) TGG TTG TTT TC | 56-58 | |
| 23S rDNA^{b,c} | NH₂-LIZ | AAG CGG CAC GGA(C/G/T) AGC TGG | 63-65 | linezolid |
| linezolid | | | | resistance |
| resistance | | | | mutations |
| determinating | | | | (G, sensitive; |
| region | | | | T, resistant |
| | | | | alleles) |
| | NegHybProbe | TCT AGA CAG CCA CTC ATA | 51 | negative |
| | | | | hybridization |
| | | | | control |
| Arabidopsis | HybProbe | GAT TGG ACG AGT CAG GAG C | 60 | positive |
| thaliana | | | | hybridization |
| | | | | control |
| | HybTarget | F*-GCT CCT GAC TCG TCC AAT C | 60 | complementary to |
| | | | | HybProbe |
| mecA | NH₂-mecA-F* | AGT TCT GCA GTA CCG GAT TTG C-F* | | Spotting control, |
| (S. aureus) | | | | orientation on |
| | | | | the array |

^{a}T_{M} was calculated with the oligonucleotide properties calculator (http://www.basic.nwu.edu/biotools/oligocalc.html).
^{b}Process controls, targeting ubiquitous PCR amplicons in both multiplex PCR reactions.
^{c}Four identical capture probes each, differing only at one central position (underlined).
^{d}E. faecium only
F* Fluoresceine-label

Oligonucleotide Array Fabrication

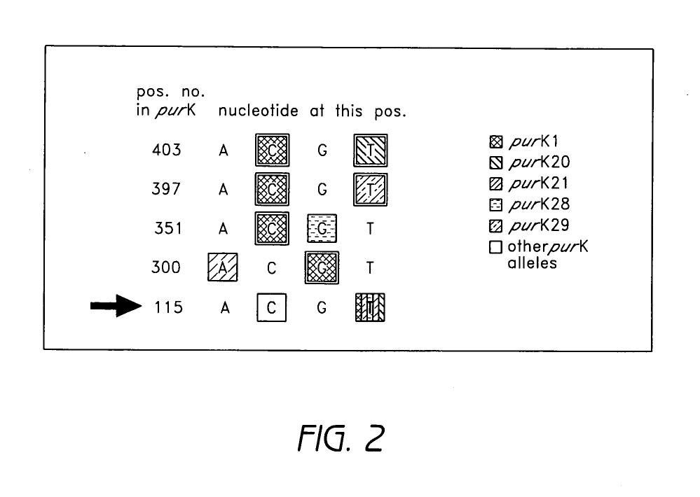

Arrays were spotted at the Institute of Technical Biochemistry, University of Stuttgart. Briefly, lyophilized oligonucleotide probes (HPLC purity grade) were dissolved in spotting buffer (160 mM Na2SO4, 130 mM Na2HPO4) to a final concentration of 20 μM and spotted using a MicroGrid II equipped with MicroSpot 2500 pins (BioRobotics, Cambridge, UK) on epoxy modified glass slides (Elipsa AG, Berlin, Germany). For covalent immobilization of the oligonucleotides the array was incubated at 60° C. for 30 minutes. All capture probes were spotted in triplicate and resulting spots had an average size of 150 //m. For a layout of the complete array see FIG. 2. Prior to hybridization slides were blocked; therefore they were rinsed for 5 minutes in washing solution I (0.1% (v/v) Triton X 100), for 4 minutes in washing solution II (0.5 μl conc. HCl per ml A. bidest.) and for 10 minutes in washing solution III (100 mM KCl) while constantly stirring. Subsequently, the slides were incubated, with the spotted side upwards, in blocking solution (25% (v/v) ethylenglycol, 0.5 μl conc. HCl per ml A. bidest.) for 20 minutes at 50° C. Finally they were rinsed in A. bidest. for 1 minute and dried by centrifugation.

PCR Amplification and Labeling

All primers were tested in single PCR amplifications with DNA from genotypically defined isolates before being used in the multiplex assay. Single PCR amplifications were performed with Ready-to-Go-PCR beads (Amersham Pharmacia Biotech, Freiburg, Germany) in a 25 μl reaction mixture containing approximately 10 ng of template DNA and 2.5 pmol of each primer. Initial denaturation at 94° C. for 3 min was followed by 35 cycles of amplification with 94° C. for 30 s, annealing at 50° C. for 30 s, and extension at 72° C. for 30 s (except for the final cycle, which had an extension step of 4 min). The PCR products were analyzed on a 1.5% agarose gel and were further controlled by sequencing.

Sequencing reactions were carried out using the ABI PRISM BigDye Terminator cycle-sequencing ready reaction kit (Applied Biosystems, Foster City, Calif.) as specified by the manufacturer. Sequence comparison to the published sequence data was performed with the DNASTAR software package (DNASTAR Inc., Madison, Wis.). Multiplex PCR amplifications were carried out in 50 μl volume reactions comprising approximately 20 ng of template DNA, a final concentration of 0.4 mM of each deoxyribonucleoside triphosphate, and 5 U of Taq DNA polymerase (Amersham Pharmacia Biotech) in 1×PCR buffer supplied by the manufacturer; the MgCl2 final concentration in the PCR mixture was adjusted to 4 mM. Cycling conditions were the same as described above. Amplification products were analyzed on a 2.5% agarose gel (80 V for 200 min) and on an Agilent Bioanalyzer together with the DNA 1000 LabChip kit (Agilent Technologies, Böblingen, Germany), respectively, to separate the different amplification products efficiently. The 13 primer pairs were divided into two multiplex PCR reactions (Table 1). In the first PCR reaction we amplified fragments of 7 different antibiotic resistance genes (ermB, vatD, vatE, vanA, vanB, aacA-aphD, aadE) and a fragment of the enterococcal 23S rDNA as amplification control using 10 pmol of all primers in the reaction. In the second PCR we amplified fragments of 2 virulence genes (esp, hyl), as well as a species specific fragment of the ddl gene and a fragment of the purK gene together with the linezolid resistance determinating region of the 23S rDNA as amplification control. To guarantee uniform amplification of all fragments the purK primer pair was used in 4-fold concentration.

Before labeling amplification products of both multiplex PCRs were pooled and purified using the QIAquick PCR purification kit (Qiagen, Hilden, Germany).

A photochemical labeling of PCR products with Psoralen-PEO-Biotin (Pierce Chemicals, Rockford, USA) was used. Briefly, 18 μl of purified PCR product was labeled in a μl reaction volume containing a final concentration of 200 μM Psoralen-PEO-Biotin. Photoreactive labeling occurred during a 30 minute exposure to long UV-light (365 nm). 20 μl of labeled multiplex PCR product (the whole labeling reaction mixture) was hybridized to the array without further purification.

Array Hybridization and Washing

Hybridization of denatured labeled PCR products was performed in 130 μl of 3×SSPE using doubled Gene Frames and appropriate cover slips (Thermo Life Science, Dreieich, Germany) in an Eppendorf thermomixer equipped with an exchangeable slides thermoblock (Eppendorf, Hamburg, Germany) for 4 hours at 45° C. with agitation (1200 rpm). To control hybridization efficiency, the hybridization mixture contained 0.25 μl of a 5′-terminal fluorescently labeled oligonucleotide complementary to the hybridization control capture probe (HybTarget, 0.05 μM, table 1). After hybridization the slides were washed with 2×SSC, 0.5% SDS, then with 1×SSC and finally with 0.1×SSC, each time for 10 minutes at room temperature, before they were dried by centrifugation. Finally, the array was incubated with 15 μl Streptavidin-Cy3 conjugate (Amersham Biosciences, Freiburg, Germany), diluted 1:500 in TBST buffer for 15 minutes under a glass coverslip.

Data Acquisition and Processing

Fluorescent images of the micro-arrays were obtained by scanning the slides with an ArrayWoRx biochip reader (Applied Precision, Marlborough, UK) using a resolution of 9.750 μm and the 590 nm filter. Fluorescence signal intensities from each spot as well as the intensity values for the local background were analyzed by use of the GeneSpotter software (MicroDiscovery, Berlin, Germany). The resulting raw data was further processed using Excel (Microsoft). For calculation of individual net signal intensities (herein to as signal intensity, SI) the local background was subtracted from the corresponding raw spot intensity values. A mean intensity value for each capture was assessed from the three replicate spots for each probe. That mean intensity value was normalized to the mean intensity value of the process control probes (the resulting value is herein referred to as relative signal intensity).

For the detection of SNPs in the 23S rDNA and the purK gene, respectively, we chose an alternative, “internal” normalization strategy according to Grimm et al. (J. Clin. Microbiol. 2000, 42:3766-3774). Within the probe set the probe with the highest mean signal intensity was considered the perfect match (PM), the remaining three probes were considered mismatches (MM). For comparison, we calculated “internal” relative signal intensities by normalizing the mean signal intensities of all SNP probes to that of the PM probe resulting in relative intensities of 1 for all PM probes and relative intensities below 1 for all MM probes.

Multiplex PCR Amplification

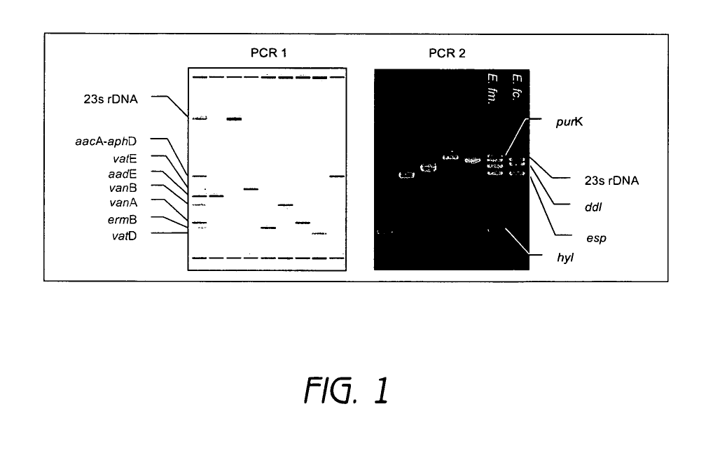

Before the optimization of the multiplex reaction, we ensured that the single PCR amplifications yielded amplicons of the expected sizes. reaction conditions for both multiplex PCR reactions were optimized using a DNA mixture (containing all targets) and following the general principles described by Henegariu et al. (Biotechniques; 1997, 23: 504-511) to amplify the 13 targets almost equally. Therefore, different concentrations of primers, oligonucleotides, and MgCl2 were tested until the optimal conditions as described above were adjusted. FIG. 1 shows an agarose gel stained with ethidium-bromide and a picture obtained from Agilent Bioanalyzer to illustrate the typical results obtained with the optimized multiplex PCR assays. To verify the identity of the PCR products, the single amplicons were sequenced; all sequences obtained were identical to those obtained from the databases. Furthermore, digoxigenin-labeled amplicons were used as probes in a southern blot hybridization. All probes hybridized to the expected fragments of the multiplex PCR amplifications, indicating that all targets were amplified efficiently and correctly in the multiplex approach (data not shown).

Selection of Capture Probes

Starting with various capture probes targeting different sites in each PCR amplicon and including sense and antisense probes, after hybridization with fluorescently labeled single PCR products, probes were checked their signal intensities and potential cross reactivity. Sequences of those oligonucleotide capture probes which were finally selected for the construction of the array are listed in Table 2. Capture probes targeting the genes ermB, vatD, vatE, vanA, vanB, aacA-aphD, aadE, 23S rDNA, and hyl (E. faecium) were selected directly from the database entries. For the esp amplicon, which is amplified from E. faecium as well as from E. faecalis a single capture probe could be selected targeting the esp gene of both species after sequence alignment (table 2). Species specific capture probes inside the single ddl amplicon were selected after sequence alignment and according to Ozawa et al. (Syst. Appl. Microbiol.; 2000, 23:230-237) [table 2]. Capture probes for the detection of linezolid resistance in E. faecium and E. faecalis, respectively, target a single nucleotide polymorphism (SNP) in the linezolid resistance determinating region of the 23S rDNA (E. faecium 6 copies, E. faecalis 4 copies). A single nucleotide transversion from guanine to uracil at position 2576 in 23S rDNA (E. coli numbering) leads to resistance with one mutated copy being sufficient for development of resistance (Werner, G. et al.; J. Clin. Microbiol., 2004, 42:5327-5331). The probe sets for SNP detection consists of 4 identical probes differing only at the central position covering the base of interest (table 2). The selected capture probe set carries an additional mismatch within the capture probe sequence to facilitate reliable discrimination between the respective alleles in homozygote and heterozygote isolates (table 2). More heterozygote isolates have to be tested to determine, whether a quantification of mutated and non-mutated loci in a single isolate will be reliable.

To detect E. faecium isolates of the highly epidemic C1 population the presence of the purK1 allele has to be investigated (Homan, W. L. et al.; J. Clin. Microbiol., 2002 40:1963-1971). The purK1 allele is characterized by a nucleotide transversion from cytosine to thymine at position 115 in the gene. This transversion can be found in 5 different purK allele types. Characterization of four additional positions inside the gene facilitates the discrimination between the 5 allele types (FIG. 2). Therefore we selected one probe sets for each of the five respective loci (Table 2). To improve the discriminatory power of four of these probe sets one or two additional mismatches had to be introduced into the capture probe sequence (Table 2).

Setting Up the System for Combined Detection of Genes and SNPs

To establish the complete procedure of multiplex pre-amplification, labeling and hybridization DNA from genotypically characterized strains was used. Different amounts of PCR products, labeling protocols and hybridization conditions were tested until the optimal conditions as described above were adjusted. Following this protocol were able to detect all genes of interest as well as all investigated SNP's in one hybridization reaction.

Testing of Clinical Isolates and Correlation to Phenotypic Antibiotic Resistance Testing

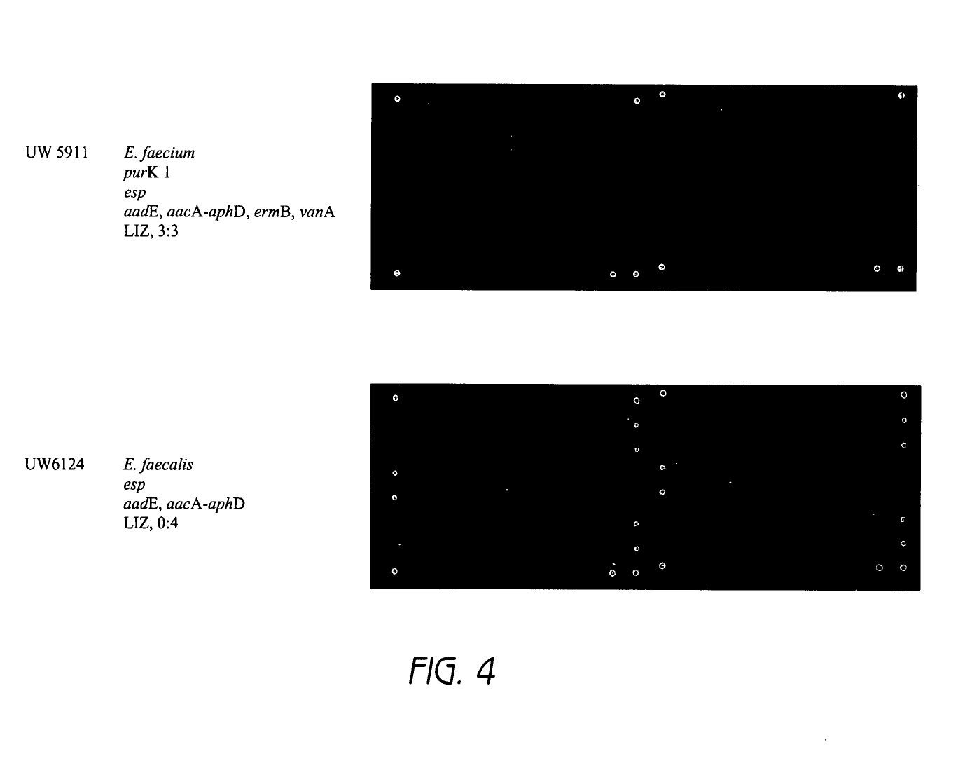

To give proof of the presented concept, currently different clinical isolates are tested and compared to the results of the microarray experiments with those obtained from PCR and phenotypical resistance testing, respectively. Hybridization experiments are conducted repeatedly. Hybridization patterns for two different isolates are shown in FIG. 4. First results reveal a high concordance between array hybridization experiments and results of PCR, sequencing and phenotypic antibiotic resistance determination, respectively.

What is claimed is:

1. A micro-array comprising a carrier and immobilized thereon in the form of a pattern nucleic acids comprising sequences specific for at least 4 target genes of bacteria of the genus Enterococcus and at least one gene characteristic for bacteria of the genus Enterococcus.

2. The micro-array according to claim 1, wherein said at least 4 target genes of bacteria of the genus Enterococcus are selected from the group consisting of aadE, aacA-aphD, ermB, vat(D), vat(E), vanA and vanB.

3. The micro-array according to claim 1, wherein said at least one gene characteristic for bacteria of the genus Enterococcus is the 23S rDNA.

4. The micro-array according to claim 1, wherein the micro-array also includes controls.

5. The micro-array according to claim 4, wherein the controls are selected from the group consisting of hyl, esp, ddl and purK.

6. The micro-array according to claim 1, wherein said carrier consists of glass, metal or plastics.

7. The micro-array according to claim 6, wherein said carrier consists or epoxy glass.

8. The micro-array according to claim 7, wherein said carrier is a microplate or a slide.

9. The micro-array according to claim 1, wherein the surface of said carrier comprises an area of at least 1 square centimetre.

10. The micro-array according to claim 1, wherein the nucleic acids are present on the carrier at a density of at least 100 molecules per square centimetre.

11. The micro-array according to claim 1, wherein said specific pattern allows the mapping of each nucleic acid to a specific position on said carrier and a specific analysis.

12. The micro-array according to claim 1, wherein said nucleic acids are immobilized via a spacer molecule.

13. A method for determining the presence of multi-resistant bacteria of the genus Enterococcus in a sample, comprising:
a) providing a micro-array comprising a carrier and immobilized thereon in the form of a pattern nucleic acids comprising sequences specific for at least 4 target genes of bacteria of the genus Enterococcus and at least one gene characteristic for bacteria of the genus Enterococcus;
b) contacting the sample with the micro-array under conditions allowing hybridization of complementary strands; and
c) determining whether hybridisation occurs.

14. The method according to claim 13, wherein said at least 4 target genes of bacteria of the genus Enterococcus are selected from the group consisting of aadE, aacA-aphD, ermB, vat(D), vat(E), vanA and vanB.

15. The micro-array according to claim 13, wherein said at least one gene characteristic for bacteria of the genus Enterococcus is 23S rDNA.

16. The method according to claim 13, wherein the micro-array also includes controls.

17. The method according to claim 16, wherein the controls are selected from the group consisting of hyl, esp, ddl and purK.

18. The method according to claim 13, wherein said sample contains nucleic acids comprising oligonucleotides and/or polynucleotides, having a length of about 10 to 100 nucleotides.

19. The method according to claim 18, wherein said oligonucleotides and/or poly-nucleotides are isolated from body tissues or fluids, particularly blood, suspected to contain bacteria of the genus Enterococcus.

20. The method according to claim 18, wherein said nucleic acids are labelled with a marker molecule.

21. The method according to claim 20, wherein said marker molecule is selected from the group consisting of cyanine dyes, preferably Cy3 and/or Cy5, renaissance dyes, preferably ROX and/or R110, and fluorescent dyes, preferably FAM and/or FITC

22. A diagnostic kit for the detection infections with bacteria of the genus Enterococcus, comprising nucleic acids specific for at least 4 target genes of bacteria of the genus Enterococcus and/or a micro-array comprising a carrier and immobilized thereon in the form of a pattern nucleic acids comprising sequences specific for at least 4 target genes of bacteria of the genus Enterococcus and at least one gene characteristic for bacteria of the genus Enterococcus; and optionally buffers.

23. The kit according to claim 20, wherein the target genes are selected from the group consisting of aadE, aacA-aphD, ermB, vat(D), vat(E), vanA and vanB.

24. The kit according to claim 20, wherein said at least one gene characteristic for bacteria of the genus Enterococcus is 23S rDNA.

25. The kit according to claim 20, wherein controls are included.

26. The method according to claim 25, wherein the controls are selected from the group consisting of hyl, esp, ddl and purK.
